(19) United States
(12) Patent Application Publication
(10) Pub. No.: US 2022/0409571 A1
(43) Pub. Date: Dec. 29, 2022

(54) CANNABIDIOL-BASED TOPICAL REMEDY

(71) Applicant: Ryan DelloRusso, Sunnyvale, CA (US)

(72) Inventor: Ryan DelloRusso, Sunnyvale, CA (US)

(21) Appl. No.: 17/846,693

(22) Filed: Jun. 22, 2022

Related U.S. Application Data
(60) Provisional application No. 63/214,532, filed on Jun. 24, 2021.

(51) Int. Cl. A61K 31/352; A61K 31/05; A61K 36/73; A61K 47/44

(52) U.S. Cl. CPC A61K 31/352; A61K 31/05; A61K 36/73; A61K 47/44

(57) ABSTRACT

A method for forming a cannabis-based topical remedy includes adding cannabis to an oil. The method further includes heating the mixture of the cannabis and the oil for a first period of time. The method further includes adding a wax to the heated mixture. The method further includes packaging a resulting mixture.

### CROSS-REFERENCE TO RELATED APPLICATIONS

This application claims the benefit and priority of U.S. Provisional Application No. 63/214,532, titled CANNABIDIOL-BASED TOPICAL REMEDY and filed on Jun. 24, 2021, the entire contents of which is hereby incorporated by reference in its entirety.

### BACKGROUND

### 1. Field

The present disclosure relates to systems and methods for forming CBD-based topical remedies, including methods utilizing cannabis flower from which THC has been previously extracted.

### 2. Description of the Related Art

The use of cannabis products (e.g., products that include cannabidiol (CBD)) has been growing in popularity and its legality has been increasing nationwide. Various cannabis-derived products have been found to provide diverse pharmacological benefits such as pain relief, anti-inflammatory, antioxidant, and antibiotic benefits. Such products may include oils used in vaporizer cartridges, edible products, topical products, and cannabis flower itself (typically smoked). Because of the recent surge in popularity and push towards legalization, the market for such cannabis-derived products has been growing rapidly. Typically, cannabis flower is treated once to extract cannabinoids for a single product (or single set of products) and is then discarded.

Thus, there is a need in the art for new cannabinoid-based products such as topical remedies.

### SUMMARY

Described herein is a method for forming a cannabis-based topical remedy. The method includes adding cannabis to an oil. The method further includes heating the mixture of the cannabis and the oil for a first period of time. The method further includes adding a wax to the heated mixture. The method further includes packaging a resulting mixture.

In any of the foregoing embodiments, the cannabis is recycled cannabis waste flower.

Any of the foregoing embodiments may further include drying the recycled cannabis waste flower prior to adding the cannabis to the oil.

Any of the foregoing embodiments may further include adding a scented compound to the mixture.

Any of the foregoing embodiments may further include grinding the cannabis into a powder prior to adding the cannabis to the oil.

Any of the foregoing embodiments may further include melting the wax into the heated mixture of the cannabis and the oil.

Any of the foregoing embodiments may further include adding an additional oil to the heated mixture during or after melting the wax into the heated mixture.

In any of the foregoing embodiments, a ratio of weight of the oil to weight of the cannabis is between 16-to-1 and 64-to-1.

In any of the foregoing embodiments, the oil includes a coconut oil.

Also disclosed is a method for forming a cannabis-based topical remedy. The method includes adding cannabis to an oil. The method further includes heating the mixture of the cannabis and the oil for a first period of time. The method further includes adding a wax to the heated mixture. The method further includes allowing the heated mixture to cool.

In any of the foregoing embodiments, the cannabis is recycled cannabis waste flower.

Any of the foregoing embodiments may further include drying the recycled cannabis waste flower prior to adding the cannabis to the oil.

Any of the foregoing embodiments may further include adding a scented compound to the mixture.

Any of the foregoing embodiments may further include grinding the cannabis into a powder prior to adding the cannabis to the oil.

Any of the foregoing embodiments may further include melting a wax into the mixture of the cannabis and the oil.

Any of the foregoing embodiments may further include adding an additional oil to the heated mixture during or after melting the wax into the heated mixture.

In any of the foregoing embodiments, a ratio of weight of the oil to weight of the cannabis is between 16-to-1 and 64-to-1.

In any of the foregoing embodiments, the oil includes a coconut oil.

In any of the foregoing embodiments, the cannabis flower is pulverized prior to being added to the oil.

Also disclosed is a method for forming a cannabis-based topical remedy. The method includes drying previously-used cannabis. The method further includes pulverizing the dried cannabis. The method further includes adding the pulverized cannabis flower to an oil. The method further includes heating the mixture of the cannabis and the oil for a first period of time. The method further includes adding a wax to the heated mixture. The method further includes packaging a resulting mixture.

### BRIEF DESCRIPTION OF THE DRAWINGS

Other systems, methods, features, and advantages of the present invention will be or will become apparent to one of ordinary skill in the art upon examination of the following figures and detailed description. It is intended that all such additional systems, methods, features, and advantages be included within this description, be within the scope of the present invention, and be protected by the accompanying claims. Component parts shown in the drawings are not necessarily to scale, and may be exaggerated to better illustrate the important features of the present invention. In the drawings, like reference numerals designate like parts throughout the different views, wherein:

FIG. 1 is a flowchart illustrating a method for forming a cannabidiol-based topical remedy using cannabis flower according to some embodiments of the present disclosure; and

FIG. 2 is a flowchart illustrating a method for forming a cannabidiol-based topical remedy using recycled cannabis waste flower according to some embodiments of the present disclosure.

### DETAILED DESCRIPTION

The present disclosure describes systems and methods for forming a cannabidiol (CBD)-based topical remedy. The topical remedy may be useful for various conditions or disorders such as to reduce pain or inflammation, moisturize skin, or the like. The system and methods used herein may advantageously make use of recycled cannabis waste flower from which tetrahydrocannabinol (THC) has been previously extracted. In that regard, the cannabis flower may be used multiple times—a first time to extract THC for use in a first type of product and a second time to create the CBD-based topical remedy described herein.

Various recipes are described herein for forming CBD-based topical remedies. One skilled in the art will realize that these recipes or methods are exemplary only and do not limit the scope of the present disclosure.

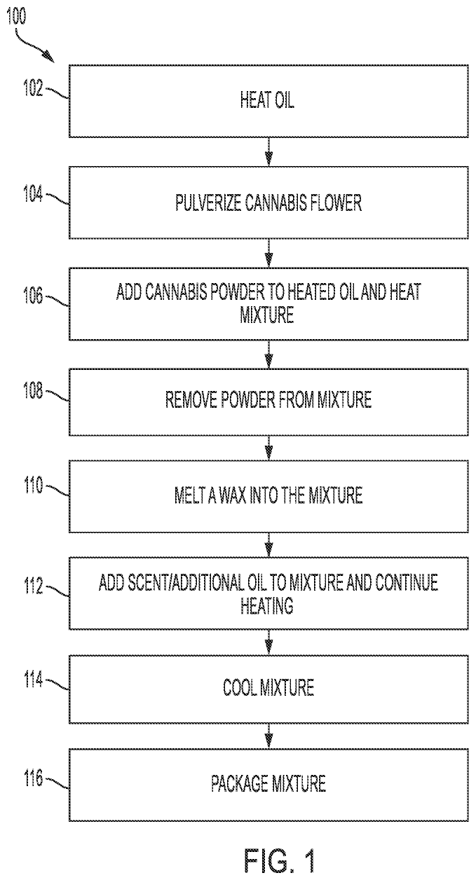

FIG. 1 illustrates a first method 100 for forming a CBD-based topical remedy. The first method 100 may be used to form a first CBD-based topical remedy using non-recycled (i.e., original) cannabis flower. This recipe may be used to form, for example, 40 ounces (oz) of the remedy. Although the methods are shown as utilizing specific ingredients at specific concentrations, one skilled in the art will realize that the specific ingredients and concentrations may be adjusted without departing from the scope of the present disclosure. The first method may include the following ingredients:

Between 24 oz and 40 oz (e.g., 32 oz (4 cups)) coconut oil

Between 0.5 oz and 1.5 oz (e.g., 1 oz) cannabis flower

Between 0.25 cups and 1 cup (e.g., ½ cup) each essential oil

Between 5 oz and 15 oz (e.g., 10 oz) white beeswax

Between 0.25 cup and 1 cup (e.g., ½ cup) olive oil

Although the method calls for coconut oil, other oils may be used in its place. For example, any one or more of the following may be substituted in place of the coconut oil: butter, olive oil, grapeseed oil, sunflower oil, avocado oil, vegetable oil, almond oil, hemp seed oil, hazelnut oil, or any other known oils. It may be desirable for these oils to be at least one of consumable-grade or non-toxic such that any undesirable side effects from topical use are minimized or avoided.

The essential oil may include any one or more essential oil, may be replaced with any other natural or artificial scented compound, or may be left out of the method for a relatively scent-free topical remedy. For example, the essential oil may include lavender essential oil, peppermint essential oil, chamomile essential oil, tea tree essential oil, frankincense essential oil, lemongrass essential oil, clary sage essential oil, or any other essential oils. In some embodiments, the essential oil(s) may be replaced with alternative fragrant compounds. For example, the essential oil(s) may be replaced with fragrance oils, concretes and absolutes, herbal infusions, dry botanicals, or any other fragrant (or non-fragrant) compound. Addition of the essential oil may add fragrance to the topical remedy to increase its market appeal.

The beeswax may include any one or more beeswax product, may be replaced with any other artificial or natural wax (e.g., soy wax, candelilla wax, paraffin wax, carnauba wax, olive wax, rice bran wax, sunflower wax, or the like), or may be left out of the method. The wax may act as an emulsifier by creating an emulsion between oily compounds and non-oily compounds (e.g., water) to resist separation of the oily compounds and non-oily compounds. The wax may further provide a relatively smooth consistency to the topical remedy, thicken the texture of the topical remedy, cause the topical remedy to feel “soft” when applied, or the like.

Any one or more oil may be used in place of, or in addition to, the olive oil. For example, any one or more of the following may be substituted in place of the olive oil: butter, coconut oil, grapeseed oil, sunflower oil, avocado oil, vegetable oil, almond oil, hemp seed oil, hazelnut oil, or any other known oils.

An exemplary method, shown as the method 100 of FIG. 1, will now be described. One skilled in the art will realize that the blocks of the method 100 may be performed in the order as shown; however, in some embodiments, some blocks may be added, some blocks may be removed, and some blocks may be performed in alternative order without departing from the scope of the present disclosure. The method 100 may begin in block 102 by heating 32 ounces of the coconut oil (or replacement) in a heating vat (e.g., a crock pot, such as size J, may be used during home processing). For example, the oil may be heated to a relatively low temperature such as between 100 and 300 degrees Fahrenheit (37.8 degrees Celsius and 149 degrees Celsius), between 150 and 250 degrees Fahrenheit (65.6 degrees Celsius and 121 degrees Celsius), between 175 and 225 degrees Fahrenheit (79.4 degrees Celsius and 107 degrees Celsius), or the like.

In block 104, the cannabis flower (e.g., 28 to 32 grams) may be pulverized or ground into a powder using a grinder (e.g., a coffee grinder may be used during home processing, a pulverizer may be used in a commercial setting, or the like). In some embodiments, the cannabis flower may be ground to a size larger than a powder without departing from the scope of the present disclosure. For example, the powder may have an average diameter of 1 inch, 0.5 inches, 0.25 inches, 0.1 inches, 0.05 inches, 0.01 inches, or the like.

In block 106, the cannabis flower powder may be added to the heated coconut oil. The mixture may then be heated for a period of time such as between 2 and 20 hours, between 5 and 15 hours, between 8 and 12 hours, or the like. The mixture may be heated at a relatively low temperature such as between 100 and 300 degrees Fahrenheit (37.8 degrees Celsius and 149 degrees Celsius), between 150 and 250 degrees Fahrenheit (65.6 degrees Celsius and 121 degrees Celsius), between 175 and 225 degrees Fahrenheit (79.4 degrees Celsius and 107 degrees Celsius), or the like.

In block 108 and after heating, the cannabis material may be filtered out of the coconut oil mixture using any filtering mechanism (e.g., by passing the coconut oil through filter paper or any other filter). The resulting coconut oil may be infused with CBD from the ground cannabis flower.

In block 110 and after removal of the cannabis material, the cannabis-infused oil may be returned to the heating vat. Beeswax (e.g., 10 ounces, or replacement thereof) may be added to the CBD-infused oil. The mixture may be heated until the wax is completely melted into the oil (e.g., at a similar temperature as above or at another temperature).

If a scent-providing compound (e.g., essential oil) is to be added, it may be introduced in block 112 after melting the wax into the oil (e.g., while the heating vat is still exposed to a heating temperature). In some embodiments, if no scent-providing compound is used then another oil may be used in place of the scent-providing compound. In some embodiments, the scent-providing compound may be added at the same time as the wax. In some embodiments, additional oil may be added with the scent-providing compound (e.g., a mix of olive oil and essential oils may be added at this step). The mixture of the scent-providing compound, the olive oil, and the cannabis-infused mixture may be heated at a relatively low temperature (e.g., between 100 and 300 degrees Fahrenheit, between 150 and 250 degrees Fahrenheit, between 175 and 225 degrees Fahrenheit, or the like) for a period of time (e.g., between 10 and 200 minutes, between 15 and 120 minutes, between 30 and 60 minutes, or the like). This heating allows the wax to create an emulsion between oily compounds and non-oily compounds (e.g., any fluid from the cannabis flower).

In block 114 and after heating the mixture, the mixture may be removed from the heat and allowed to cool slightly (while ensuring the oil remains in liquid form). Stated differently, the mixture may be allowed to cool to a temperature that causes the mixture to remain in liquid form without likelihood of damaging or deforming any container into which it will be placed. In some embodiments, the mixture may be placed into a container without cooling, or may be placed into a container after full cooling to room temperature has occurred.

In block 116, the partially-cooled mixture may be placed into an application unit of choice (e.g., a jar, a deodorant roll-on stick, a Tupperware container, a tube dispenser, or any other container). The mixture may be placed into the application unit using any technique. For example, the mixture may be poured through a funnel into the container. As another example, the mixture may be poured from the heating vessel into the container. As yet another example, a manufacturing line may include an automatic filling feature to automatically fill the vessel with the mixture. The mixture may be allowed to continue cooling in its container of choice either at room temperature or in a cooling unit (e.g., in a refrigerator).

Although specific measurements of the ingredients are provided, the disclosure contemplates departures from the specific weight percentages of the specific recipes. The measurement of each ingredient may be increased or decreased by 5 percent, 10 percent, 25 percent, 50 percent, or the like. For example, the coconut oil may be provided in a weight that is between 30.4 and 33.6 ounces, between 28.8 and 35.2 ounces, between 24 and 40 ounces, or the like. In addition, some ingredients may be eliminated from the recipe (e.g., essential oils may be left out of the topical remedy).

Although the recipe is provided for making a specific amount of topical remedy, one skilled in the art will realize that the amounts may be adjusted without departing from the scope of the present disclosure. For example, in order to form 80 ounces of the topical remedy, the quantities disclosed above may be doubled (because the quantities disclosed above are sufficient to form 40 ounces of the topical remedy, the quantities of ingredients may be doubled to double the output).

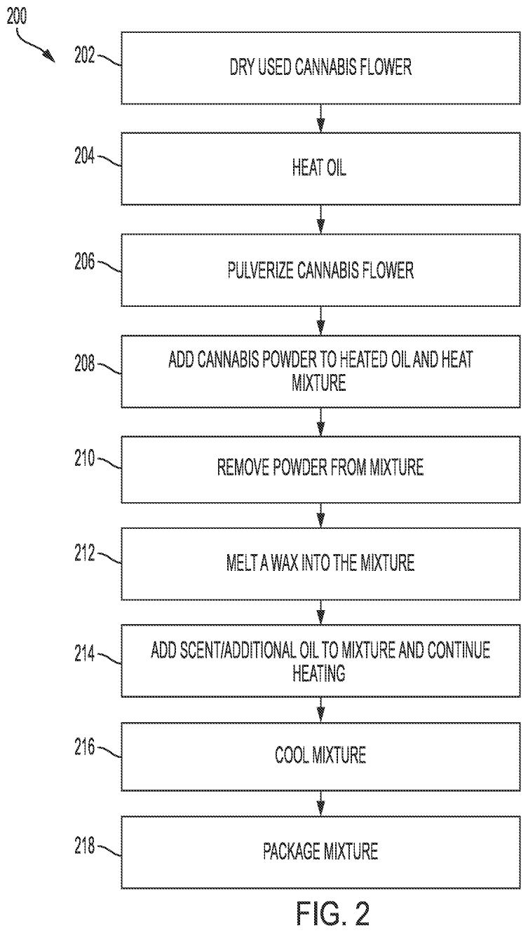

Referring now to FIG. 2, a second method 200 (discussed below) may be used to form a topical remedy. Although the first method may use recycled cannabis waste flower in place of the cannabis flower, the second method is specifically designed to maximize use of recycled cannabis waste flower. That is, the second method is specifically formulated to utilize recycled cannabis waste flower in place of fresh cannabis flower. In that regard, THC may be extracted from fresh cannabis flower to manufacture THC products (e.g., edible THC products, THC oil products (e.g., vaporizer cartridges), THC topical applications, or the like). The cannabis flower may be utilized after the THC extraction to form a topical remedy. Thus, the method 200 may utilize the cannabis for a second product after a first product or set of products have been produced using the THC from the cannabis flower.

Before performing the method 200, the THC may be removed from the cannabis flower using any known technique. For example, the THC may be removed using water extraction (plunge frozen cannabis flower into cold water, stir the plant until the trichomes fall off, and pass the water through a mesh screen or bag). As another example, the THC may be removed using isopropyl oil extraction (soak dried cannabis flower in isopropyl alcohol, agitate the cannabis flower in the container/alcohol, filter the liquid through a mesh, and remove the solvent from the liquid). As yet another example, the THC may be removed using CO2 extraction (pass hot supercritical CO2 (carbon dioxide in a liquid state) through cannabis flower, put the liquid through a separator to remove CO2 gas to produce a THC-infused liquid that is free of toxins).

The second method 200 may utilize the following ingredients:

Between 24 oz and 40 oz (e.g., 32 oz (4 cups)) coconut oil

Between 0.5 oz and 6 oz (e.g., between 1-4 oz) dried recycled cannabis waste flower (in some embodiments, the cannabis waste flower may be provided as a wet or frozen biomass; the weight range provided above may be a weight of the cannabis waste when dried out)

Between 0.25 cup and 1 cup (e.g., ½ cup) each essential oil

Between 5 oz and 15 oz (e.g., 10 oz) white beeswax

Between 0.25 cup and 1 cup (e.g., ½ cup) olive oil

Any replacement ingredients discussed above with reference to the first method 100 of FIG. 1 may likewise be substituted in the second method 200. For example, any other oil may be used in place of the coconut oil. The recycled cannabis waste flower may be provided as a wet product, as a frozen product (with or without included added moisture), or the like. For example, the cannabis flower may be used to form hashish and, as a result, may retain moisture from the process. In that regard, the method may include drying the recycled cannabis waste flower in block 202. This may be performed in any known method of drying biomass such as heating the biomass (e.g., between 125 and 275 degrees Fahrenheit, between 150 and 250 degrees Fahrenheit, between 175 and 225 degrees Fahrenheit, or the like). The heating may be performed, for example, in a convection oven. The biomass may be heated for a period of time such as between 10 and 200 minutes, between 15 and 150 minutes, between 30 and 120 minutes, or the like. The biomass may be stirred while heating. The biomass should be monitored during the heating and drying process to ensure it fails to burn. The biomass may then finish drying at room temperature such as by placing on a rack, in a drying net, on a countertop, or the like. It is acceptable for the recycled cannabis waste flower to be lightly toasted but should retain a shade of green. The process of heating the recycled cannabis flower reactivates the cannabinoids that remain in the recycled flower. In that regard, it may be desirable to dry the biomass using heat, as described above. However, the biomass may be dried using any other known method such as hang drying at room temperature, freeze drying, or the like.

In block 204, after drying the biomass, 32 ounces of the coconut oil (or replacement) may be placed in a heating vat and heated (e.g., the heating vat may include a crock pot, such as size J, during home processing). For example, the oil may be heated to a relatively low temperature such as between 100 and 300 degrees Fahrenheit (37.8 degrees Celsius and 149 degrees Celsius), between 150 and 250 degrees Fahrenheit (65.6 degrees Celsius and 121 degrees Celsius), between 175 and 225 degrees Fahrenheit (79.4 degrees Celsius and 107 degrees Celsius), or the like.

In block 206, the dried, recycled cannabis flower (e.g., 0.5 oz and 6 oz) may be pulverized or ground into a powder using a grinder (e.g., a coffee grinder may be used during home processing, a pulverizer may be used in a commercial setting, or the like). In some embodiments, the cannabis flower may be ground to a size larger than a powder without departing from the scope of the present disclosure. For example, the powder may have an average diameter of 1 inch, 0.5 inches, 0.25 inches, 0.1 inches, 0.05 inches, 0.01 inches, or the like.

In block 208, the cannabis flower powder may be added to the heated coconut oil. The mixture may then be heated for a period of time such as between 2 and 20 hours, between 5 and 15 hours, between 8 and 12 hours, or the like. The mixture may be heated at a relatively low temperature such as between 100 and 300 degrees Fahrenheit (37.8 degrees Celsius and 149 degrees Celsius), between 150 and 250 degrees Fahrenheit (65.6 degrees Celsius and 121 degrees Celsius), between 175 and 225 degrees Fahrenheit (79.4 degrees Celsius and 107 degrees Celsius), or the like.

In block 210 and after heating, the cannabis material may be filtered out of the coconut oil mixture using any filtering mechanism (e.g., by passing the coconut oil through filter paper or any other filter). The resulting coconut oil may be infused with CBD from the ground cannabis flower.

In block 212 and after removal of the cannabis material, the cannabis-infused oil may be returned to the heating vat. Beeswax (e.g., 10 ounces, or replacement thereof) may be added to the CBD-infused oil. The mixture may be heated until the wax is completely melted into the oil (e.g., at a similar temperature as above or at another temperature).

If a scent-providing compound (e.g., essential oil) is to be added, it may be introduced in block 214 after melting the wax into the oil (e.g., while the heating vat is still exposed to a heating temperature). In some embodiments, if no scent-providing compound is used then another oil may be used in place of the scent-providing compound. In some embodiments, the scent-providing compound may be added at the same time as the wax. In some embodiments, additional oil may be added with the scent-providing compound (e.g., a mix of olive oil and essential oils may be added at this step). The mixture of the scent-providing compound, the olive oil, and the cannabis-infused mixture may be heated at a relatively low temperature (e.g., between 100 and 300 degrees Fahrenheit, between 150 and 250 degrees Fahrenheit, between 175 and 225 degrees Fahrenheit, or the like) for a period of time (e.g., between 10 and 200 minutes, between 15 and 120 minutes, between 30 and 60 minutes, or the like). This heating allows the wax to create an emulsion between oily compounds and non-oily compounds (e.g., any fluid from the cannabis flower).

In block 216 and after heating the mixture, the mixture may be removed from the heat and allowed to cool slightly (while ensuring the oil remains in liquid form). Stated differently, the mixture may be allowed to cool to a temperature that causes the mixture to remain in liquid form without likelihood of damaging or deforming any container into which it will be placed. In some embodiments, the mixture may be placed into a container without cooling, or may be placed into a container after full cooling to room temperature has occurred.

In block 218, the partially-cooled mixture may be placed into an application unit of choice (e.g., a jar, a deodorant roll-on stick, a Tupperware container, a tube dispenser, or any other container). The mixture may be placed into the application unit using any technique. For example, the mixture may be poured through a funnel into the container. As another example, the mixture may be poured from the heating vessel into the container. As yet another example, a manufacturing line may include an automatic filling feature to automatically fill the vessel with the mixture. The mixture may be allowed to continue cooling in its container of choice either at room temperature or in a cooling unit (e.g., in a refrigerator).

Although specific measurements of the ingredients are provided, the disclosure contemplates departures from the specific weight percentages of the specific recipes. The measurement of each ingredient may be increased or decreased by 5 percent, 10 percent, 25 percent, 50 percent, or the like. For example, the coconut oil may be provided in a weight that is between 30.4 and 33.6 ounces, between 28.8 and 35.2 ounces, between 24 and 40 ounces, or the like. In addition, some ingredients may be eliminated from the recipe (e.g., essential oils may be left out of the topical remedy).

Although the recipe is provided for making a specific amount of topical remedy, one skilled in the art will realize that the amounts may be adjusted without departing from the scope of the present disclosure. For example, in order to form 80 ounces of the topical remedy, the quantities disclosed above may be doubled (because the quantities disclosed above are sufficient to form 40 ounces of the topical remedy, the quantities of ingredients may be doubled to double the output).

The present disclosure provides methods for creating a CBD-infused topical remedy using cannabis flower. The disclosure provides additional benefits such as providing methods for creating a CBD-infused product using cannabis flower from which THC has already been extracted. The topical remedies formed using the described methods may provide benefits such as reduced acne, an anti-aging effects, reduced pain and inflammation, reduced stress and tension, or the like.

Where used throughout the specification and the claims, “at least one of A or B” includes “A” only, “B” only, or “A and B.” Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A method for forming a cannabis-based topical remedy, the method comprising:
adding cannabis to an oil;
heating the mixture of the cannabis and the oil for a first period of time;
adding a wax to the heated mixture; and
packaging a resulting mixture.

2. The method of claim 1, wherein the cannabis is recycled cannabis waste flower.

3. The method of claim 2, further comprising drying the recycled cannabis waste flower prior to adding the cannabis to the oil.

4. The method of claim 1, further comprising adding a scented compound to the mixture.

5. The method of claim 1, further comprising grinding the cannabis into a powder prior to adding the cannabis to the oil.

6. The method of claim 1, further comprising melting the wax into the heated mixture of the cannabis and the oil.

7. The method of claim 6, further comprising adding an additional oil to the heated mixture during or after melting the wax into the heated mixture.

8. The method of claim 1, wherein a ratio of weight of the oil to weight of the cannabis is between 16-to-1 and 64-to-1.

9. The method of claim 1, wherein the oil includes a coconut oil.

10. A method for forming a cannabis-based topical remedy, the method comprising:
adding cannabis to an oil;
heating the mixture of the cannabis and the oil for a first period of time;
adding a wax to the heated mixture; and
allowing the heated mixture to cool.

11. The method of claim 10, wherein the cannabis is recycled cannabis waste flower.

12. The method of claim 11, further comprising drying the recycled cannabis waste flower prior to adding the cannabis to the oil.

13. The method of claim 10, further comprising adding a scented compound to the mixture.

14. The method of claim 10, further comprising grinding the cannabis into a powder prior to adding the cannabis to the oil.

15. The method of claim 10, further comprising melting a wax into the mixture of the cannabis and the oil.

16. The method of claim 15, further comprising adding an additional oil to the heated mixture during or after melting the wax into the heated mixture.

17. The method of claim 10, wherein a ratio of weight of the oil to weight of the cannabis is between 16-to-1 and 64-to-1.

18. The method of claim 10, wherein the oil includes a coconut oil.

19. The method of claim 10, wherein the cannabis flower is pulverized prior to being added to the oil.

20. A method for forming a cannabis-based topical remedy, the method comprising:
drying previously-used cannabis;
pulverizing the dried cannabis;
adding the pulverized cannabis flower to an oil;
heating the mixture of the cannabis and the oil for a first period of time;
adding a wax to the heated mixture; and
packaging a resulting mixture.
